(19) United States
(12) Patent Application Publication
(10) Pub. No.: US 2019/0119362 A1
(43) Pub. Date: Apr. 25, 2019

(54) METHOD FOR PRODUCING PROTEIN INCLUDING k CHAIN VARIABLE REGION

(71) Applicant: KANEKA CORPORATION, Osaka (JP)

(72) Inventors: Shinichi Yoshida, Hyogo (JP); Dai Murata, Hyogo (JP)

(73) Assignee: KANEKA CORPORATION, Osaka (JP)

(21) Appl. No.: 16/176,090

(22) Filed: Oct. 31, 2018

Related U.S. Application Data
(63) Continuation of application No. PCT/JP2017/015500, filed on Apr. 17, 2017.

(30) Foreign Application Priority Data
May 6, 2016 (JP) 2016-093457

(51) Int. Cl. C07K 16/06; C07K 1/22

(52) U.S. Cl. CPC C07K 16/065; C07K 2317/56; C07K 1/22

(57) ABSTRACT

A method for producing a protein includes adsorbing a protein including a κ chain variable region on an insoluble carrier of an affinity separation matrix by contacting a liquid sample including the protein with the affinity separation matrix; washing the affinity separation matrix to remove impurities; separating the protein from the affinity separation matrix by using an acidic buffer; and regenerating the affinity separation matrix by using an alkaline aqueous solution after the protein is separated from the affinity separation matrix. The insoluble carrier includes a ligand immobilized on the insoluble carrier, and the ligand is a κ chain variable region-binding peptide including B5 domain of Protein L derived from Peptostreptococcus magnus 312 strain or a variant of the B5 domain. The adsorbing, the washing, and the separating are repeated 3 or more times.

### TECHNICAL FIELD

One or more embodiments of the present invention relate to a method for efficiently producing a protein containing a κ chain variable region.

### BACKGROUND

As one of important functions of a protein, an ability to specifically bind to a specific molecule is exemplified. The function plays an important role in an immunoreaction and signal transduction in a living body. A technology utilizing the function for purifying a useful substance has been actively developed. As one example of proteins which are actually utilized industrially, for example, Protein A affinity separation matrix has been used for capturing an antibody drug to be purified with high purity at one time from a culture of an animal cell (Non-patent documents 1 and 2). Hereinafter, Protein A is abbreviated as “SpA” in some cases.

An antibody drug which has been developed is mainly a monoclonal antibody, and a monoclonal antibody has been produced on a large scale by using recombinant cell cultivation technology. A “monoclonal antibody” means an antibody obtained from a clone derived from a single antibody-producing cell. Most of antibody drugs which are presently launched are classified into an immunoglobulin G (IgG) subclass in terms of a molecular structure. In addition, an antibody drug consisting of an antibody derivative such as an antibody fragment has been actively subjected to clinical development. An antibody fragment has a molecular structure obtained by fragmenting an immunoglobulin, and various antibody fragment drugs have been clinically developed (Non-patent Document 3).

In an initial purification step of an antibody drug production process, the above-described SpA affinity separation matrix is utilized. SpA is, however, basically a protein which specifically binds to a Fc region of IgG. Thus, SpA affinity separation matrix cannot capture an antibody fragment which does not contain a Fc region. Accordingly, an affinity separation matrix capable of capturing an antibody fragment which does not contain a Fc region of IgG is highly required industrially in terms of a platform development of a process for purifying an antibody drug.

A plurality of peptides which bind to a region except for a Fc region of IgG have been already known (Non-patent Document 4). Among such peptides, a peptide which can bind to a variable region as an antigenic determinant region may be most preferred in terms of many kinds of antibody fragment format to be bound and an ability to also bind to IgM and IgA. As such a peptide, for example, Protein L has been well-known. Hereinafter, Protein L is abbreviated as “PpL” in some cases. PpL is a protein which contains a plurality of κ-chain variable region-binding domains, and amino acid sequences of each K-chain variable region-binding domain are different from each other. Hereinafter, a κ-chain variable region is abbreviated as “VL-κ” in some cases. In addition, the number of VL-κ-binding domains and amino acid sequences of each VL-κ-binding domain are different depending on the kind of a strain. For example, the number of VL-κ-binding domains in PpL of Peptostreptococcus magnus 312 strain is 5, and the number of VL-κ-binding domains in PpL of Peptostreptococcus magnus 3316 strain is 4 (Non-patent documents 5 to 7, and Patent documents 1 and 2). There are no domains that have the same amino acid sequence as each other in the totally 9 VL-κ-binding domains.

A plurality of affinity separation matrixes having PpL as a ligand have been commercially available. In the case of SpA, a study has been undertaken using a recombinant peptide in which a plurality of one kind of specific antibody-binding domain are linked to each other as a ligand (Non-patent document 1). On the one hand, in the case of PpL, there has been little study on a difference in physical properties and functions due to a difference in the amino acid sequences of individual VL-κ-binding domains and there remains room for improvement.

### PATENT DOCUMENT

Patent Document 1: JP H7-506573 T
Patent Document 2: JP H7-507682 T

### Non-Patent Document

Non-patent Document 1: Hober S., et al., J. Chromatogr. B, 2007, vol. 848, pp. 40-47
Non-patent Document 2: Shukla A. A., et al., Trends Biotechnol., 2010, vol. 28, pp. 253-261
Non-patent Document 3: Nelson A. N., et al., Nat. Biotechnol., 2009, vol. 27, pp. 331-337
Non-patent Document 4: Bouvet P. J., Int. J. Immunopharmac., 1994, vol. 16, pp. 419-424
Non-patent Document 5: Kastern W., et al., J. Biol. Chem., 1992, vol. 267, pp. 12820-12825
Non-patent Document 6: Murphy J. P., et al., Mol. Microbiol., 1994, vol. 12, pp. 911-920
Non-patent Document 7: Housden N. G., et al., Biochemical Society Transactions, 2003, vol. 31, pp. 716-718

When an antibody and an antibody fragment are purified, a carrier is produced by immobilizing a protein containing an antibody-binding domain as a ligand, a column is filled with the carrier, and the antibody and antibody fragment are selectively captured on the ligand. The antibody and antibody fragment adsorbed on the ligand are eluted by flowing an acidic aqueous solution, and the ligand is regenerated by flowing an alkaline aqueous solution.

A general protein is denatured by an alkaline aqueous solution, and the function cannot be often maintained. Also, an antibody-binding domain immobilized on a carrier cannot be regenerated by an alkaline aqueous solution or cannot tolerate repeated use in some cases.

One or more embodiments of the present invention provide a method for efficiently producing a protein containing a κ chain variable region without frequent replacement of an affinity separation matrix by using the specific κ chain variable region-binding peptide having an excellent resistance to an alkali as a ligand.

### SUMMARY

The inventors intensively studied and completed one or more embodiments of the present invention by finding that B5 domain of Protein L derived from Peptostreptococcus magnus 312 strain or the variant thereof is excellent in an alkali resistance, and it becomes possible that an affinity separation matrix is regenerated by an alkaline aqueous solution and a protein containing a κ chain variable region is efficiently purified without frequent replacement of the affinity separation matrix by using the B5 domain or variant thereof as a ligand.

Hereinafter, one or more embodiments of the present invention are described.

[1] A method for producing a protein containing a κ chain variable region, comprising the steps of

First step: contacting a liquid sample containing the protein with an affinity separation matrix in order to adsorb the protein on an insoluble carrier, wherein a κ chain variable region-binding peptide containing B5 domain of Protein L derived from Peptostreptococcus magnus 312 strain or a variant of the B5 domain is immobilized as a ligand on the insoluble carrier in the affinity separation matrix,

Second step: washing the affinity separation matrix to remove an impurity except for the protein,

Third step: separating the protein on the affinity separation matrix from the affinity separation matrix by using an acidic buffer, and

Fourth step: regenerating the affinity separation matrix by using an alkaline aqueous solution after the protein is separated from the affinity separation matrix,

wherein the First step through the Third step are repeated 3 or more times.

[2] The method according to the above [1], wherein the Fourth step is also repeated 3 or more times after the Third step.

[3] The method according to the above [1] or [2], wherein an amino acid sequence of the B5 domain or the variant is any one of the following amino acid sequences:

(1) an amino acid sequence of SEQ ID NO: 7 or SEQ ID NO: 16;

(2) an amino acid sequence of SEQ ID NO: 7 or SEQ ID NO: 16 with deletion, substitution and/or addition of 1 or more and 10 or less amino acid residues, wherein the amino acid sequence has a binding ability to the κ chain variable region;

(3) an amino acid sequence having a sequence identity of 85% or more with the amino acid sequence of SEQ ID NO: 7 or SEQ ID NO: 16, wherein the amino acid sequence has a binding ability to the κ chain variable region.

[4] The method according to the above [3], wherein an amino acid sequence of the variant of the B5 domain corresponds to an amino acid sequence of SEQ ID NO: 7 wherein the 17^{th} position is Glu, the 19^{th} position is Ile, the 20^{th} position is Tyr, the 22^{nd} position is Glu, the 25^{th} position is Thr, the 26^{th} position is Val, the 30^{th} position is Thr, the 50^{th} position is Ser and the 53^{rd} position is His.

[5] The method according to the above [3], wherein an amino acid sequence of the variant of the B5 domain corresponds to an amino acid sequence of SEQ ID NO: 16 wherein the 7^{th} position is Glu, the 9^{th} position is Ile, the 10^{th} position is Tyr, the 12^{th} position is Glu, the 15^{th} position is Thr, the 16^{th} position is Val, the 20^{th} position is Thr, the 40^{th} position is Ser and the 43^{rd} position is His.

[6] The method according to any one of the above [3] to [5], wherein a multimer of the B5 domain or the variant having the amino acid sequence is immobilized as the ligand on the insoluble carrier.

The κ chain variable region-binding activity of the affinity separation matrix according to one or more embodiments of the present invention is decreased only slightly due to damage by an alkaline treatment, since the affinity separation matrix is produced by immobilizing the κ chain variable region-binding peptide containing the specific Protein L domain or variant thereof as a ligand. Thus, when the affinity separation matrix is repeatedly used, the matrix can be washed by a sodium hydroxide aqueous solution having high concentration for a long time. As a result, an impurity such as an organic substance remaining on a chromatography carrier can be effectively removed. In addition, it is amazing that B5 domain of Protein L derived from Peptostreptococcus magnus 312 strain used in one or more embodiments of the present invention has the above-described properties, since the constructs consisting of B1 to B4 domain are mainly studied in Patent document 1 and Non-patent document 4.

### BRIEF DESCRIPTION OF THE DRAWINGS

FIG. 1 is a figure to show a method for producing an expression vector of LB5t-Wild.1d.

FIG. 2 is a graph on which a logarithm of an affinity constant (K_{A}), an association rate constant (k_{ON}) or a dissociation rate constant (k_{OFF}) of various VL-κ-binding domains derived from PpL to various IgG-Fab are plotted.

FIG. 3 are chromatography charts by applying a polyclonal Fab to LB5t-Wild.4d-immobilized carrier or commercially available Protein L carrier and then eluting the Fab with an elution buffer and a strong washing buffer.

FIG. 4 is a magnified figure of a part of the chromatography charts of FIG. 3, at which the polyclonal Fab was applied.

FIG. 5 is a graph on which various peptide concentrations and a binding response used for evaluating residual binding activities of various VL-κ-binding domains derived from PpL to aRSV-Fab are plotted.

FIG. 6 is a graph to show residual binding activities of various VL-κ-binding domains derived from PpL to aRSV-Fab after an alkaline treatment.

### DETAILED DESCRIPTION OF THE EMBODIMENTS

Hereinafter, each step according to one or more embodiments of the present invention is described.

First Step: Step for Adsorbing Target Protein

In the present step, a liquid sample containing a protein containing a κ chain variable region is contacted with an affinity separation matrix in order to adsorb the protein on an insoluble carrier, wherein a κ chain variable region-binding peptide containing B5 domain of Protein L derived from Peptostreptococcus magnus 312 strain or a variant thereof is immobilized as a ligand on the insoluble carrier in the affinity separation matrix.

An “immunoglobulin (Ig)” is a glycoprotein produced by a B cell of a lymphocyte and has a function to recognize a specific molecule such as a protein to be bound. An immunoglobulin has not only a function to specifically bind to a specific molecule referred to as antigen but also a function to detoxify and remove an antigen-containing factor in cooperation with other biological molecule or cell. An immunoglobulin is generally referred to as “antibody”, and the name is inspired by such functions.

All of immunoglobulins basically have the same molecular structure. The basic structure of an immunoglobulin is a Y-shaped four-chain structure. The four-chain structure is composed of two light chains and two heavy chains of polypeptide chains. A light chain (L chain) is classified into two types of λ chain and κ chain, and all of immunoglobulins have either of the chains. A heavy chain (H chain) is classified into five types of γ chain, μ chain, a chain, δ chain and a chain, and an immunoglobulin is classified into an isotype depending on the kind of a heavy chain. An immunoglobulin G (IgG) is a monomer immunoglobulin, is composed of two γ chains and two light chains, and has two antigen-binding sites.

A lower half vertical part in the “Y” shape of an immunoglobulin is referred to as a “Fc region”, and an upper half “V” shaped part is referred to as a “Fab region”. A Fc region has an effector function to initiate a reaction after an antibody binds to an antigen, and a Fab region has a function to bind to an antigen. A Fab region of a heavy chain and a Fc region are bound to each other through a hinge part. Papain, which is a proteolytic enzyme and which is contained in papaya, decomposes a hinge part to cut into two Fab regions and one Fc region. The part close to the tip of the “Y” shape in a Fab region is referred to as a “variable region (V region)”, since there are various changes of the amino acid sequence in order to bind to various antigens. A variable region of a light chain is referred to as a “VL region”, and a variable region of a heavy chain is referred to as a “VH region”. A Fab region except for a V region and a Fc region are referred to as a “constant region (C region)”, since there is relatively less change. A constant region of a light chain is referred to as a “CL region”, and a constant region of a heavy chain is referred to as a “CH region”. A CH region is further classified into three regions of CH1 to CH3. A Fab region of a heavy chain is composed of a VH region and CH1, and a Fc region of a heavy chain is composed of CH2 and CH3. There is a hinge part between CH1 and CH2. Protein L binds to a variable region of which light chain is κ chain (VL-κ) (Non-patent Documents 5 to 7).

The κ chain variable region-binding peptide which is immobilized as a ligand on a carrier in one or more embodiments of the present invention binds to a κ chain variable region (VL-κ) of an immunoglobulin. In one or more embodiments, the VL-κ-containing protein to which the peptide binds contains at least VL-κ, and may be IgG containing a Fab region and a Fc region without deficiency, or other Ig series such as IgM, IgD and IgA, or a derivative of an immunoglobulin molecule prepared by a protein engineering mutation. An immunoglobulin molecule derivative to which the VL-κ-binding peptide of one or more embodiments of the present invention binds is not particularly restricted as long as the derivative contains VL-κ. For example, the immunoglobulin molecule derivative is exemplified by a Fab fragment prepared by fragmenting immunoglobulin G into a Fab region only, scFv consisting of only a variable region of immunoglobulin G, chimeric immunoglobulin G prepared by replacing a part of human immunoglobulin G domains with an immunoglobulin G domain of other organism to be fused, immunoglobulin G of which sugar chain in the Fc region is mutated, and a scFv fragment to which a drug is covalently bound.

The term “peptide” in one or more embodiments of the present invention means any molecules having a polypeptide structure. In the range of the “peptide”, not only a so-called protein but also a fragmented protein and a protein to which other peptide is bound through a peptide bond are included. In one or more embodiments of the present invention, the terms “protein” and “peptide” are conveniently used to clearly distinguish a VL-κ-containing protein from a VL-κ-binding peptide, but a peptide is substantively used synonymously with a protein. The term “domain” means a unit of higher-order structure of a protein. A domain is composed of from dozens to hundreds of amino acid residues, and means a peptide unit which can sufficiently serve some kind of a physicochemical or biochemical function. The term “variant” of a protein or peptide means a protein or peptide obtained by introducing at least one substitution, addition or deletion of an amino acid into a sequence of a wild protein or peptide. A mutation to substitute an amino acid is described by adding a wild or non-mutated amino acid residue before the number of a substituted position and adding a mutated amino acid residue after the number of the substituted position. For example, the mutation to substitute Gly at the 29^{th} position by Ala is described as G29A.

In the present First step, a liquid sample containing a VL-κ-containing protein is contacted with the affinity separation matrix in which the specific ligand is immobilized in order to selectively adsorb the VL-κ-containing protein. The liquid sample is not particularly restricted as long as the liquid sample contains a VL-κ-containing protein to be purified, and in one or more embodiments, it is preferred that the liquid sample is a solution in which a VL-κ-containing protein is dissolved in an aqueous solvent. Such a liquid sample is exemplified by a serum sample which contains a VL-κ-containing protein, and a homogenate of a hybridoma which produces a monoclonal antibody.

The affinity matrix usable in one or more embodiments of the present invention contains an insoluble carrier and a ligand. The “insoluble carrier” used in one or more embodiments of the present invention shows insolubility to an aqueous solvent used for a protein solution, and when a ligand is immobilized on the insoluble carrier, the peptide which specifically binds to the ligand can be purified.

The insoluble carrier usable in one or more embodiments of the present invention is exemplified by an inorganic carrier such as glass beads and silica gel; an organic carrier composed of a synthetic polymer such as cross-linked polyvinyl alcohol, cross-linked polyacrylate, cross-linked polyacrylamide and cross-linked polystyrene; an organic carrier composed of a polysaccharide such as crystalline cellulose, cross-linked cellulose, cross-linked agarose and cross-linked dextran; and a composite carrier obtained by the combination of the above carriers, such as an organic-organic composite carrier and an organic-inorganic composite carrier. The commercially available product thereof is exemplified by porous cellulose gel GCL2000, Sephacryl S-1000 prepared by crosslinking allyl dextran and methylene bisacrylamide through a covalent bond, an acrylate carrier Toyopearl, a cross-linked agarose carrier Sepharose CL4B, and a cross-linked cellulose carrier Cellufine. It should be noted, however, that the insoluble carrier usable in one or more embodiments of the present invention is not restricted to the carriers exemplified as the above.

In one or more embodiments, it is preferred that the insoluble carrier usable in one or more embodiments of the present invention has large surface area and is porous with a large number of fine pores having a suitable size in terms of a purpose and a method of the use of the affinity separation matrix. The carrier may have any form such as beads, monolith, fiber and film including hollow fiber, and any form can be selected.

The term “ligand” in one or more embodiments of the present invention means a substance and a functional group to selectively bind to a target molecule from an aggregate of molecules on the basis of a specific affinity between molecules, such as binding between an antigen and an antibody, and means the peptide which specifically binds to VL-κ. In one or more embodiments of the present invention, the term “ligand” also means an “affinity ligand”.

In one or more embodiments, the peptide is utilized as an affinity ligand having an affinity for an immunoglobulin or a fragment thereof, particularly VL-κ. An affinity separation matrix prepared by immobilizing the ligand on an insoluble carrier is similarly included in one or more embodiments of the present invention.

The ligand used in one or more embodiments of the present invention is a κ chain variable region-binding peptide containing B5 domain of Protein L derived from Peptostreptococcus magnus 312 strain or a variant of the B domain. The “variant” means the above-described B5 domain having a VL-κ-binding ability with deletion, substitution and/or addition of 1 or more amino acid residues in the amino acid sequence. In one or more embodiments, the number of the mutation is preferably not more than 20 or not more than 15, more preferably not more than 10 or not more than 8, and even more preferably not more than 5 or not more than 3.

The “Protein L” (PpL) is a protein derived from a cell wall of anaerobic gram-positive coccus in the genus of Peptostreptococcus. In one or more embodiments of the present invention, PpL is preferably derived from Peptostreptococcus magnus, and preferably two kinds of PpL derived from Peptostreptococcus magnus 312 strain and Peptostreptococcus magnus 3316 strain. In one or more embodiments, PpL derived from 312 strain is preferably used. In one or more embodiments of the present invention, in some cases, the PpL derived from Peptostreptococcus magnus 312 strain is abbreviated as “PpL 312”, and the PpL derived from Peptostreptococcus magnus 3316 strain is abbreviated as “PpL 3316”. The amino acid sequence of PpL 312 is shown as SEQ ID NO: 1, and the amino acid sequence of PpL 3316 is shown as SEQ ID NO: 2, which also contain a signal sequence.

PpL contains a plurality of VL-κ-binding domains having 70 to 80 residues in the protein molecule. The number of VL-κ-binding domains contained in PpL 312 is 5, and the number of VL-κ-binding domains contained in PpL 3316 is 4. The VL-κ-binding domains contained in PpL 312 are referred to as B1 domain (SEQ ID NO: 3), B2 domain (SEQ ID NO: 4), B3 domain (SEQ ID NO: 5), B4 domain (SEQ ID NO: 6), and B5 domain (SEQ ID NO: 7) in the order from the N-terminal, and the VL-κ-binding domains contained in PpL 3316 are referred to as C1 domain (SEQ ID NO: 8), C2 domain (SEQ ID NO: 9), C3 domain (SEQ ID NO: 10), and C4 domain (SEQ ID NO: 11) in the order from the N-terminal (Non-patent documents 5 and 6). The amino acid sequence of B5 domain of PpL 312 used in one or more embodiments of the present invention is preferably the amino acid sequence of SEQ ID NO: 7.

It has been found from a research that about 20 residues of the VL-κ-binding domain at the N-terminal part do not form a specific secondary structure; and even when the N-terminal part is deleted, the three-dimensional structure and the VL-κ-binding property of the VL-κ-binding domain are maintained (Non-patent Document 7). For example, peptides having the amino acid sequence of SEQ ID NO: 12 with respect to B1 domain, the amino acid sequence of SEQ ID NO: 13 with respect to B2 domain, the amino acid sequence of SEQ ID NO: 14 with respect to B3 domain, the amino acid sequence of SEQ ID NO: 15 with respect to B4 domain, the amino acid sequence of SEQ ID NO: 16 with respect to B5 domain, the amino acid sequence of SEQ ID NO: 17 with respect to C1 domain, the amino acid sequence of SEQ ID NO: 18 with respect to C2 domain, the amino acid sequence of SEQ ID NO: 19 with respect to C3 domain, and the amino acid sequence of SEQ ID NO: 20 with respect to C4 domain also function as a VL-κ-binding domain. It is preferred that the amino acid sequence of B5 domain of PpL 312 used in one or more embodiments of the present invention is the amino acid sequence of SEQ ID NO: 16, which corresponds to the amino acid sequence of SEQ ID NO: 7 with deletion of the N-terminal region and the C-terminal region.

The amino acid sequence of SEQ ID NO: 1 and/or SEQ ID NO: 2 with deletion of several residues at the N-terminal and/or the C-terminal is included in one or more embodiments of the present invention. In one or more embodiments, the number of residues to be deleted is preferably 1 or more and 5 or less, more preferably 1 or more and 4 or less, even more preferably 1 or more and 3 or less, even more preferably 1 or 2, and even more preferably 1.

In one or more embodiments of the present invention, the phrase, a peptide “has a (specific) amino acid sequence”, means that the specific amino acid sequence is contained in the amino acid sequence of the peptide and the function of the peptide is maintained. The sequence of the peptide other than a specific amino acid sequence is exemplified by a signal peptide, a histidine tag, a linker sequence for immobilization, and a crosslinking structure such as —S—S-bond. The amino acid sequence of the peptide may be naturally the same as a specific amino acid sequence.

As one of the embodiments, a fusion peptide characterized in that the VL-κ-binding peptide of one or more embodiments of the present invention is fused as one component with other peptide having a different function is exemplified. The other peptide is exemplified by albumin, GST, i.e. glutathione S-transferase, or a histidine tag, but is not restricted to the examples. In addition, peptides fused with a nucleic acid such as DNA aptamer, a drug such as an antibiotic or a polymer such as PEG, i.e. polyethylene glycol, are also included in one or more embodiments of the present invention as long as the availability of the peptide according to one or more embodiments of the present invention is utilized in such a fusion peptide.

For example, the amino acid sequence of the above-described B5 domain used in one or more embodiments of the present invention is specifically exemplified by the following amino acid sequences (1) to (3):

(1) an amino acid sequence of SEQ ID NO: 7 or SEQ ID NO: 16;

(2) an amino acid sequence of SEQ ID NO: 7 or SEQ ID NO: 16 with deletion, substitution and/or addition of 1 or more and 10 or less amino acid residues, wherein the amino acid sequence has a binding ability to κ chain variable region;

(3) an amino acid sequence having a sequence identity of 85% or more with the amino acid sequence of SEQ ID NO: 7 or SEQ ID NO: 16, wherein the amino acid sequence has a binding ability to a κ chain variable region.

In the above-described amino acid sequence (2), the number of the deletion and the like of an amino acid may be preferably not more than 8, not more than 6 or not more than 5, more preferably not more than 4 or not more than 3, even more preferably 2 or less, and particularly preferably 1.

In the above-described amino acid sequence (3), the term “sequence identity” in the phrase of “amino acid sequence having a sequence identity of 85% or more with the amino acid sequence identified in the (1)” is not particularly restricted as long as the peptide having such an amino acid sequence identity shows a binding ability to a κ chain variable region. The amino acid sequence identity is not particularly restricted as long as the identity is 85% or more, and may be preferably not less than 86%, not less than 88′ or not less than 90%, more preferably not less than 92%, not less than 94% or not less than 95%, even more preferably not less than 96%, not less than 98% or not less than 99%, and particularly preferably not less than 99.5% or not less than 99.8%. The term “sequence identity” in one or more embodiments of the present invention means an identity degree of an amino acid residue between 2 or more amino acid sequences. When an identity between certain two amino acid sequences is higher, an identity and a homology between the sequences are higher. Whether 2 kinds of amino acid sequences show a specific identity or not can be analyzed by directly comparing the sequences, specifically by using a program for amino acid sequence multiple alignment, such as Clustal (http://www.clustal.org/omega/) and a commercially available sequence analysis software.

In the above-described amino acid sequences (2) and (3), the phrase “having a binding ability to a κ chain variable region” means, for example, a binding ability to a κ chain variable region can be detected in an affinity test for IgG-Fab by using a biosensor in Example 2(2) described later.

Specifically, the amino acid sequence of the B5 domain used in one or more embodiments of the present invention is preferably exemplified by an amino acid sequence of SEQ ID NO: 7 wherein the 17^{th} position is Glu, the 19^{th} position is Ile, the 20^{th} position is Tyr, the 22^{nd} position is Glu, the 25^{th} position is Thr, the 26^{th} position is Val, the 30^{th} position is Thr, the 50^{th} position is Ser and the 53^{rd} position is His, and an amino acid sequence of SEQ ID NO: 16 wherein the 7^{th} position is Glu, the 9^{th} position is Ile, the 10^{th} position is Tyr, the 12^{th} position is Glu, the 15^{th} position is Thr, the 16^{th} position is Val, the 20^{th} position is Thr, the 40^{th} position is Ser and the 43^{rd} position is His.

PpL is a protein in which 4 or 5 VL-κ-binding domains are linked in tandem. The VL-κ-binding peptide of one or more embodiments of the present invention, therefore, may be a monomer or a multimer composed of the 2 or more, preferably 3 or more, even more preferably 4 or more, and even more preferably 5 or more monodomains. In one or more embodiments, with respect to the upper limit of the number of the domains to be linked, 10 or less is exemplified, 8 or less is preferred, and 6 or less is more preferred. The multimer may be a homomultimer in which one kind of the VL-κ-binding peptides are linked, such as homodimer and homotrimer, or a heteromultimer in which B1 to B4 domains of PpL 312 and two or more kinds of the VL-κ-binding domains are linked, such as heterodimer and heterotrimer.

A method for connecting monomer peptides of one or more embodiments of the present invention is exemplified by a connecting method through one or more amino acid residues and a direct connecting method without an amino acid residue, but is not restricted thereto. The number of the amino acid residue for connection is not particularly restricted, and may be preferably 20 residues or less, more preferably 15 residues or less, even more preferably 10 or less, even more preferably 5 or less, and even more preferably 2 or less. In one or more embodiments, it is preferred that the amino acid residue for connection does not destabilize a three dimensional structure of the monomer protein.

The VL-κ-binding peptide used as a ligand in one or more embodiments of the present invention can be prepared by an ordinary method. Specifically, DNA which encodes an amino acid sequence of a desired VL-κ-binding peptide or a fragment thereof is chemically synthesized, DNA which encodes the VL-κ-binding peptide is amplified by PCR, and the amplified DNA is inserted into a vector. Escherichia coli or the like is infected by the obtained vector and cultivated, and the desired VL-κ-binding peptide is purified from the cultivated bacteria body or a culture liquid by chromatography or the like.

In the affinity separation matrix of one or more embodiments of the present invention, the ligand is immobilized on the insoluble carrier.

The above-described ligand is covalently immobilized on the insoluble carrier directly or through a linker group. The linker group is exemplified by a C₁₋₆ alkylene group, an amino group (—NH—), an ether group (—O—), a carbonyl group (—C(═O)—), an ester group (—C(═O)—O— or —O—C(═O)—), an amide group (—C(═O)—NH— or —NH—C(═O)—), a urea group (—NHC(═O) NH—); a group formed by binding 2 or more and 10 or less groups selected from the group consisting of a C₁₋₆ alkylene group, an amino group, an ether group, a carbonyl group, an ester group, an amide group and a urea group; and a C₁₋₆ alkylene group having a group selected from the group consisting of an amino group, an ether group, a carbonyl group, an ester group, an amide group and a urea group at one end or both ends. In one or more embodiments, the above-described number of the bound groups is preferably not more than 8 or not more than 6, more preferably 5 or less, and even more preferably 4 or less. The above-described C₁₋₆ alkylene group may be substituted by a substituent such as a hydroxy group.

With respect to a method for immobilizing the ligand, for example, the ligand can be bound to a carrier by a conventional coupling method utilizing an amino group, a carboxy group or a thiol group of the ligand. Such a coupling method is exemplified by an immobilization method comprising an activation of a carrier by a reaction with cyanogen bromide, epichlorohydrin, diglycidyl ether, tosyl chloride, tresyl chloride, hydrazine, sodium periodate or the like, or introduction of a reactive functional group on the carrier surface, and the coupling reaction between the resulting carrier and a compound to be immobilized as a ligand; and an immobilization method by condensation and crosslinking which method comprises the step of adding a condensation reagent such as carbodiimide or a reagent having a plurality of functional groups in the molecule, such as glutaraldehyde, into a mixture containing a carrier and a compound to be immobilized as a ligand.

A spacer molecule may be introduced between the ligand and carrier. Alternatively, the ligand may be directly immobilized on the carrier. Accordingly, the VL-κ-binding peptide of one or more embodiments of the present invention may be chemically modified for immobilization, or may have an additional amino acid residue useful for immobilization. Such an amino acid useful for immobilization is exemplified by an amino acid having a functional group useful for a chemical reaction for immobilization in a side chain, and specifically exemplified by Lys having an amino group in the side chain and Cys having a thiol group in the side chain. In one or more embodiments of the present invention, since the binding ability of the peptide according to one or more embodiments of the present invention to VL-K is principally maintained in a matrix prepared by immobilizing the peptide as a ligand, and any modification and change for immobilization are included in one or more embodiments of the present invention.

In the present First step, a liquid sample containing a VL-κ-containing protein is contacted with the affinity separation matrix in order to adsorb the target protein on the insoluble carrier. In one or more embodiments, the liquid sample is preferably a solution in which a VL-κ-containing protein is dissolved in an aqueous solvent. In addition, it may be preferred that the liquid sample is approximately neutral and the pH value thereof is 6 or more and 8 or less. The solvent of the liquid sample may be water only, may contain a water-miscible organic solvent such as C₁₋₄ alcohol as long as the liquid sample contains water as a main component, and may be a buffer solution of which pH is 6 or more and 8 or less.

In the present First step, for example, a column is filled with the affinity separation matrix to obtain an affinity column, and a liquid sample is flown through the affinity column to selectively adsorb a VL-κ-containing protein on the VL-κ-binding peptide.

Second Step: Step for Washing Affinity Separation Matrix

In the present step, the affinity separation matrix on which a VL-κ-containing protein is adsorbed in the above-described First step is washed to remove an impurity except for the target VL-κ-containing protein. Even after the present step, the VL-κ-containing protein is adsorbed on the affinity separation matrix in the column. The affinity separation matrix of one or more embodiments of the present invention is excellent in the absorption and retention performance of a target VL-κ-containing protein from the step of adding a liquid sample through the step of washing the matrix.

As a washing liquid usable for washing the affinity separation matrix in the present Second step, a washing liquid which does not disturb interaction between the VL-κ-containing protein and the VL-κ-binding peptide is used. For example, only a buffer of which pH is 6 or more and 8 or less can be used as the washing liquid.

Third Step: Step for Separating VL-κ-Containing Protein

In the present step, the VL-κ-containing protein is separated from the affinity separation matrix on which the VL-κ-containing protein is adsorbed by using an acidic buffer. By the present Third step, the purified VL-κ-containing protein can be obtained.

In the present Third step, the pH of an acidic buffer used for separating the VL-κ-containing protein from the affinity separation matrix may be appropriately adjusted, and for example, can be adjusted to about 2.0 or more and 4.0 or less. Into the acid buffer used for eluting the VL-κ-containing protein, a substance for promoting dissociation from the matrix may be added.

Forth Step: Step for Regenerating Affinity Separation Matrix

In the present step, the affinity separation matrix which is used in the above-described Third step and from which the VL-κ-containing protein is separated is regenerated by washing with an alkaline aqueous solution. It is not needed to necessarily perform the present Forth step after the above-described Third step, and the present step may be performed once every three iterations of the above First to Third steps, once every five iterations, or once every ten iterations.

The “alkaline aqueous solution” usable for the regeneration of the affinity separation matrix means an aqueous solution which exhibits alkalinity to the extent that a purpose such as washing and sterilization can be achieved. More specifically, a sodium hydroxide aqueous solution of not less than 0.01 M and not more than 1.0 M or not less than 0.01 N and not more than 1.0 N is exemplified, but the alkaline aqueous solution is not restricted thereto. In the case of sodium hydroxide, the lower limit of the concentration may be preferably 0.01 M, more preferably 0.02 M, and even more preferably 0.05 M. On the one hand, the upper limit of sodium hydroxide concentration may be preferably 1.0 M, more preferably 0.5 M, even more preferably 0.3 M, even more preferably 0.2 M, and even more preferably 0.1 M. The alkaline aqueous solution is not necessarily a sodium hydroxide aqueous solution, and in one or more embodiments, the pH thereof is preferably 12 or more and 14 or less. With respect to the lower limit of the pH, 12.0 or more is preferred, and 12.5 or more is more preferred. In one or more embodiments, with respect to the upper limit of the pH, 14 or less is preferred, 13.5 or less is more preferred, and 13.0 or less is even more preferred.

A general protein becomes denatured in an alkaline condition. A peptide used for purifying an antibody or an antibody fragment is no exception, and some peptide cannot be regenerated by an alkaline aqueous solution or the function thereof is remarkably impaired by an alkaline aqueous solution. On the one hand, the VL-κ-binding peptide used as a ligand in one or more embodiments of the present invention is excellent in a chemical stability to an alkaline aqueous solution and can be sufficiently regenerated by an alkaline aqueous solution. The term “chemical stability” generally means that a protein retains the function against a chemical modification such as a chemical change of an amino acid residue and a chemical denaturation such as a transfer and a cleavage of an amide bond. The phrase “a protein retains a function” in one or more embodiments of the present invention means that the protein retains a binding activity to VL-κ. Thus, when the “chemical stability” is higher, the degree of decrease in the binding activity to VL-K is smaller even after the protein is immersed into an alkaline aqueous solution. A binding activity to VL-K can be evaluated by a ratio of a peptide of which affinity to a VL-κ-containing protein is maintained without a chemical denaturation due to an alkaline aqueous solution as an indicator. In this disclosure, the term “resistance to alkali” has the same meaning as “chemical stability under an alkaline condition”.

The time to treat the affinity separation matrix by an alkaline aqueous solution after the above-described Third step is not particularly restricted and may be appropriately adjusted, since a damage degree of the peptide is different depending on the concentration of the alkaline aqueous solution and the temperature at the treatment. For example, when the concentration of sodium hydroxide is 0.05 M and the temperature during immersion is atmospheric temperature, the lower limit of the time to immerse the affinity separation matrix into the alkaline aqueous solution may be preferably 1 hour, more preferably 2 hours, more preferably 4 hours, more preferably 10 hour, and more preferably 20 hours, but is not particularly restricted.

As described above, since the VL-κ-binding peptide of one or more embodiments of the present invention is excellent in the chemical stability to an alkaline aqueous solution, the VL-κ-binding peptide can be regenerated by an alkaline aqueous solution after a VL-κ-containing protein is purified. In addition, even if such a regeneration treatment is repeated several times, the binding ability of the VL-κ-binding peptide to a VL-κ-containing protein is hardly decreased. Thus, according to one or more embodiments of the present invention, it becomes possible to efficiently purify a VL-κ-containing protein since it is not needed to frequently exchange the affinity separation matrix.

The present application claims the benefit of the priority date of Japanese patent application No. 2016-93457 filed on May 6, 2016. All of the contents of the Japanese patent application No. 2016-93457 filed on May 6, 2016, are incorporated by reference herein.

### EXAMPLES

Hereinafter, one or more embodiments of the present invention are described in more detail with Examples; however, the present invention is not restricted to the following Examples.

The variant peptide obtained in the following Examples is described as “peptide name—introduced mutation”, and wild type peptide, into which mutation is not introduced, is described as “peptide name—Wild”. For example, B5 domain of wild PpL 312 having SEQ ID NO: 7 is described as “LB5-Wild”. With respect to a protein formed by linking a plurality of single domains, “d” is described after the number of the linked single domains and a single domain is described as “1d”. In the following Examples, the B5 domain of PpL 312 of which N-terminal region is deleted and which has SEQ ID NO: 16 was used, since the N-terminal region does not form a secondary structure. The B5 domain is described as “LB5t-Wild” for distinguishing from SEQ ID NO: 7.

### Example 1: Preparation of B5 Domain of PpL 312 with Deletion of N-Terminal Region (LB5t-Wild.1d)

(1) Preparation of Expression Plasmid

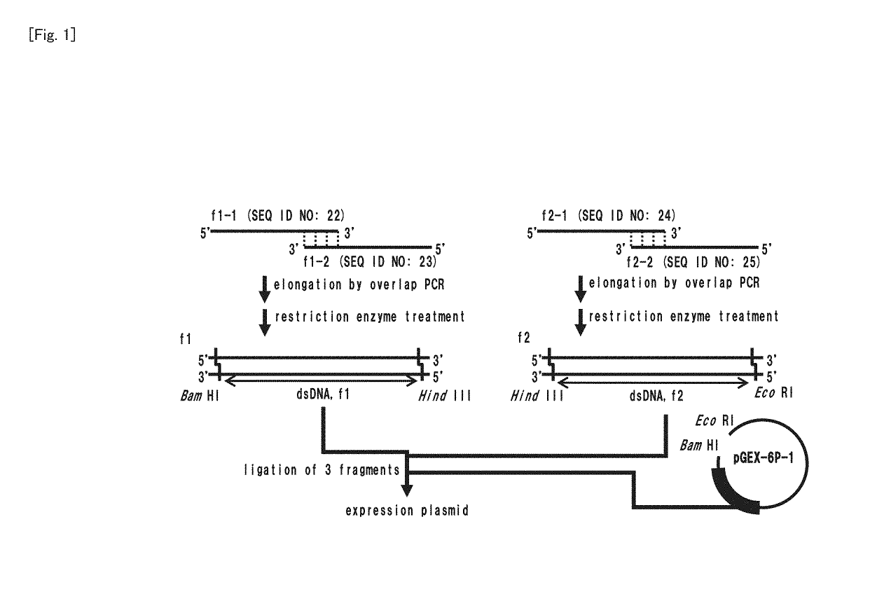

A base sequence of SEQ ID NO: 21 encoding the peptide having the amino acid sequence of LB5t-Wild.1d (SEQ ID NO: 16) was designed by reverse translation from the amino acid sequence. The method for producing the expression plasmid is shown in FIG. 1. A DNA encoding LB5t-Wild.1d was prepared by ligating two kinds of double-stranded DNAs (f1 and f2) having the same restriction enzyme site, and integrated into the multiple cloning site of an expression vector. In fact, the preparation of the peptide-encoding DNA and the integration into the vector were simultaneously performed by three fragments ligation for connecting totally three kinds of double-stranded DNAs of the two kinds of double-stranded DNAs and an expression vector. The two kinds of double-stranded DNAs were prepared by elongating two kinds of single-stranded oligo DNAs (f1−1/f1−2 or f2−1/f2−2) respectively containing about 30-base complementary region with overlapping PCR. Hereinafter, the specific experimental procedure is described. Single-stranded oligo DNAs f1−1 (SEQ ID NO: 22)/f1−2 (SEQ ID NO: 23) were synthesized by outsourcing to Sigma Genosys. The overlapping PCR was performed by using Pyrobest (manufactured by Takara Bio, Inc.) as a polymerase. The PCR product was subjected to agarose electrophoresis and the target band was cut out to extract the double-stranded DNA. The thus extracted double-stranded DNA was cleaved with the restriction enzymes BamHI and HindIII (both available from Takara Bio, Inc.). Similarly, single-stranded oligo DNAs f2−1 (SEQ ID NO: 24)/f2−2 (SEQ ID NO: 25) were synthesized by outsourcing. The double-stranded DNA synthesized by overlapping PCR was extracted and cleaved with the restriction enzymes HindIII and EcoRI (both available from Takara Bio, Inc.). Then, the two kinds of double-stranded DNAs were sub-cloned into the BamHI/EcoRI site in the multiple cloning site of a plasmid vector pGEX-6P-1 (GE Healthcare Bioscience). The ligation reaction for the subcloning was performed by using Ligation high (manufactured by TOYOBO CO., LTD.) in accordance with the protocol attached to the product.

A competent cell (“Escherichia coli HB101” manufactured by Takara Bio, Inc.) was transformed by using the above-described plasmid vector pGEX-6P-1 in accordance with the protocol attached to the competent cell product. By using the plasmid vector pGEX-6P-1, LB5t-Wild.1d which was fused with glutathione-S-transferase (hereinafter, abbreviated as “GST”) could be produced. Then, the plasmid DNA was amplified and extracted by using a plasmid purification kit (“Wizard Plus SV Minipreps DNA Purification System” manufactured by Promega) in accordance with the standard protocol attached to the kit. The base sequence of the peptide-encoding DNA of the expression plasmid was determined by using a DNA sequencer (“3130xl Genetic Analyzer” manufactured by Applied Biosystems). The sequencing PCR was performed by using a gene analysis kit (“BigDye Terminator v. 1.1 Cycle Sequencing Kit” manufactured by Applied Biosystems) and DNA primers for sequencing the plasmid vector pGEX-6P-1 (manufactured by GE Healthcare Bioscience) in accordance with the attached protocol. The sequencing product was purified by using a plasmid purification kit (“BigDye XTerminator Purification Kit” manufactured by Applied Biosystems) in accordance with the attached protocol and used for the base sequence analysis.

(2) Production and Purification of Peptide

The transformant which was produced by integrating the modified LB5t-Wild.1d gene and which was obtained in the above-described (1) was cultivated in 2xYT medium containing ampicillin at 37° C. overnight. The culture liquid was inoculated in 2xYT medium containing about 100-fold amount of ampicillin for cultivation at 37° C. for about 2 hours. Then, isopropyl-1-thio-β-D-galactoside, which is hereinafter abbreviated to “IPTG”, was added so that the final concentration thereof became 0.1 mM, and the transformant was further cultivated at 37° C. for 18 hours.

After the cultivation, the bacterial cell was collected by centrifugation and re-suspended in 5 mL of PBS. The cell was disrupted by sonication and centrifuged to separate a supernatant fraction as a cell-free extract and an insoluble fraction. When a target gene is integrated into the multiple cloning site of pGEX-6P-1 vector, a fusion peptide having GST added to the N-terminal is produced. Each fraction was analyzed by SDS electrophoresis; as a result, a peptide band assumed to be induced by IPTG was detected at a position corresponding to a molecular weight of about 25,000 or more in the cases of each of all the cell-free extracts obtained from each culture solutions of the transformant.

The GST fusion peptide was roughly purified from each of the cell-free extract containing the GST fusion peptide by affinity chromatography using a GSTrap FF column (GE Healthcare Bioscience), which has an affinity for GST. Specifically, each of the cell-free extract was added to the GSTrap FF column and the column was washed with a standard buffer (20 mM NaH₂PO₄—Na₂HPO₄, 150 mM NaCl, pH 7.4). Then, the target GST fusion peptide was eluted by using an elution buffer (50 mM Tris-HCl, 20 mM Glutathione, pH 8.0).

When a gene is integrated into the multiple cloning site of pGEX-6P-1 vector, an amino acid sequence by which GST can be cleaved by using sequence-specific protease: PreScission Protease (manufactured by GE Healthcare Bioscience) is inserted between GST and a target protein. By using such PreScission Protease, GST was cleaved in accordance with the attached protocol. The target peptide was purified by gel filtration chromatography using a Superdex 75 10/300 GL column (manufactured by GE Healthcare Bioscience) from the GST-cleaved sample used for assay. Each of the reaction mixture was added to the Superdex 75 10/300 GL column equilibrated with the standard buffer, and the target peptide therein was separated and purified from the cleaved GST and PreScission Protease.

All of the above-described peptide purification by chromatography using the column were performed by using AKTAprime plus system (manufactured by GE Healthcare Bioscience). In addition, after the cleavage of GST, the sequence of Gly-Pro-Leu-Gly-Ser derived from the vector pGEX-6P-1 was added at the N-terminal side of the peptide produced in the present example.

### Example 2: Evaluation of Affinity of LB5t-Wild.1d for IgG-Fab

(1) Preparation of Fab Fragment Derived from IgG (IgG-Fab)

Humanized monoclonal IgG drug products shown in Table 1 as raw materials were fragmented into a Fab fragment and a Fc fragment by using papain, and only Fab was purified. In this experiment, totally 6 kinds of Fab were prepared. With respect to each Fab, the name, the humanized monoclonal IgG drug product as a raw material and the like are collectively shown in Table 1.

TABLE 1

| | | | | |
|---|---|---|---|---|
| Whole | Prepared Fab name: | aRSV-Fab | aTNFa-Fab | aEGFR-Fab |
| antibody | General name: | Palivizumab | Infliximab | Cetuximab |
| (raw | Product name: | Synagis | Remicade | Erbitux |
| material) | Maker: | AbbVie | Mitsubishi Tanabe Pharma | Merck Serono |
| | Basic structure: | Humanized antibody/IgG1κ | Chimeric antibody/IgG1κ | Chimeric antibody/IgG1κ |
| | Binding part to PpL: | Human VLκ sub: 1 | Mouse VLκ sub: 5 | Mouse VLκ sub: 5 |
| Whole | Prepared Fab name: | aHER2-Fab | aIgE-Fab | aRANKL-Fab |
| antibody | General name: | Trastuzumab | Omalizumab | Denosumab |
| (raw | Product name: | Herceptin | Xolair | Pralia |
| material) | Maker: | Chugai pharmaceutical | Novartis | Daiichi sankyo |
| | Basic structure: | Humanized antibody/IgG1κ | Humanized antibody/IgG1κ | Humanized antibody/IgG2κ |
| | Binding part to PpL: | Human VLκ sub: 1 | Human VLκ sub: 1 | Human VLκ sub: 3 |

Hereinafter, a method for preparing IgG-Fab derived from anti-RSV monoclonal antibody (general name: “palivizumab”) is specifically described. In this disclosure, when the other IgG-Fab was used for evaluation, the IgG-Fab was basically prepared in a similar method. Specifically, the humanized monoclonal IgG product was dissolved in a buffer for papain digestion (0.1 M AcOH—AcONa, 2 mM EDTA, 1 mM cysteine, pH 5.5). Agarose on which papain was immobilized (“Papain Agarose from papaya latex” manufactured by SIGMA) was added thereto. The mixture was incubated at 37° C. for about 8 hours while the mixture was stirred by a rotator. The reaction mixture containing both of Fab fragment and Fc fragment was separated from the agarose on which papain was immobilized. The IgG-Fab was obtained as a flow-through fraction by affinity chromatography using MabSelect SuRe column manufactured by GE Healthcare Bioscience from the reaction mixture. The obtained IgG-Fab solution was subjected to purification by gel filtration chromatography using Superdex 75 10/300 GL column to purify IgG-Fab. The standard buffer was used for equilibrating and separation. The above peptide purification by chromatography was performed by using AKTAavant 25 system as Example 1.

(2) Analysis of Affinity of LB5t-Wild.1d for IgG-Fab

The affinity of LB5t-Wild.1d obtained in Example 1(2) for totally 6 kinds of IgG-Fab was analyzed by using a biosensor Biacore 3000 (manufactured by GE Healthcare Bioscience) utilizing surface plasmon resonance. In the present Example, the IgG-Fab obtained in Example 2(1) was immobilized on a sensor tip, and each of the peptide was flown on the tip to detect the interaction between the two. The IgG-Fab was immobilized on a sensor tip CM5 by amine coupling method using N-hydroxysuccinimide (NHS) and N-ethyl-N′-(3-dimethylaminopropyl)carbodiimide hydrochloride (EDC), and ethanolamine was used for blocking. All of the sensor tip and reagents for immobilization were manufactured by GE Healthcare Bioscience. The IgG-Fab solution was diluted to about 10 times by using a buffer for immobilization (10 mM CK₃COOH—CH₃CONa, pH 4.5), and the Fab was immobilized on the sensor tip in accordance with the protocol attached to the Biacore 3000. In addition, a reference cell as negative control was also prepared by activating another flow cell on the tip with EDC/NHS and then immobilizing human serum albumin manufactured by Wako Pure Chemical Corporation. LB5t-Wild.1d was dissolved in a running buffer (20 mM NaH₂PO₄—Na₂HPO₄, 150 mM NaCl, 0.005% P-20, pH 7.4) to prepare a protein solution having a concentration of 0.01 μM, 0.1 μM, 1 μM or 10 μM, and the protein solution was added to the sensor tip in a flow rate of 40 μL/min for 1 minute. Binding response curves at the time of addition (association phase, for 1 minute) and after the addition (dissociation phase, for 1 minute) were sequentially obtained at a measurement temperature of 25° C. After each measurement, about 20 mM NaOH was added for washing. With respect to the obtained binding response curve from which the binding response curve of the reference cell was subtracted, a fitting analysis was conducted by 1:1 binding model using a software included in the system, “BIA evaluation”, and an affinity constant (K_{A}=kₒₙ/k_{off}) for human IgG-Fab was calculated. The analysis result is shown in Table 2.

TABLE 2

| LB5t-Wild.1d | kₒₙ (×10⁵ [M⁻¹s]) | k_{off} (×10⁻² [s⁻¹]) | K_{A} (×10⁶ [M⁻¹]) |
|---|---|---|---|
| aRSV-Fab | 31.2 | 1.7 | 188.0 |
| aTNFa-Fab | 1.5 | 21.5 | 0.7 |
| aEGFR-Fab | 2.3 | 10.4 | 2.2 |
| aHER2-Fab | 5.4 | 1.0 | 56.4 |
| aIgE-Fab | 14.5 | 0.3 | 443.4 |
| aRANKL-Fab | 0.8 | 8.9 | 1.0 |

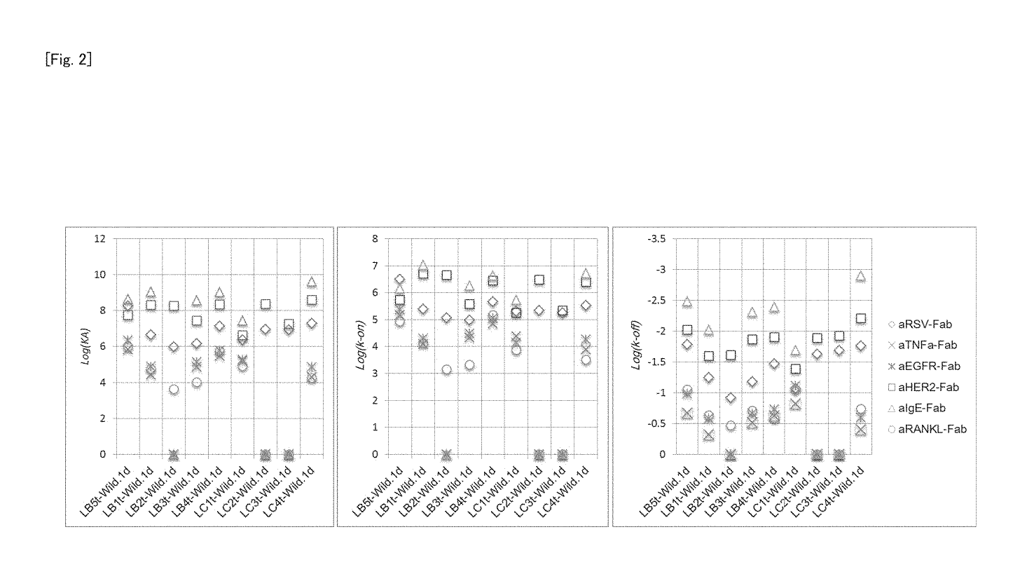

From the result with the analysis result of Comparative example 2, graphs to compare the binding abilities to IgG-Fab between the VL-κ-binding domains were prepared and are shown as FIG. 2.

As the results shown in Table 2, FIG. 2, and Tables 3 and 4 described later, LB5t-Wild.1d has higher binding ability to some Fab, such as aRSV-Fab, aTNFa-Fab and the like, than the other domains. Such a fact has not been known so far and can be said to be a surprising result. In one or more embodiments, the peptide exhibits the strongest binding ability among the tested peptides to a kind of Fab of which binding ability is wholly weak and cannot be detected to some VL-κ-binding domains, such as aTNFa-Fab and aEGFR-Fab. By such a result, a kind of Fab to which an affinity separation matrix on which a ligand on the basis of B5 domain is immobilized can be applied can be expanded.

### Comparative Example 1: Preparation of Other VL-κ-Binding Domain of PpL with Deletion of N-Terminal Region

To compare with LB5t-Wild.1d, which was B5 domain of PpL 312 with a deletion of the N-terminal, the constructs of B1 to B4 and C1 to C4 domains with a deletion of the N-terminal region were prepared. The name and amino acid sequence number of each construct are LB1t-Wild.1d (SEQ ID NO: 12), LB2t-Wild.1d (SEQ ID NO: 13), LB3t-Wild.1d (SEQ ID NO: 14), LB4t-Wild.1d (SEQ ID NO: 15), LC1t-Wild.1d (SEQ ID NO: 17), LC2t-Wild.1d (SEQ ID NO: 18), LC3t-Wild.1d (SEQ ID NO: 19) and LC4t-Wild.1d (SEQ ID NO: 20). With respect to each of the construct, each protein solution was prepared by preparing an expression plasmid and transformant, cultivating the transformant, and by purification similarly to the method of Example 1. With respect to a restriction enzyme, a restriction enzyme except for HindIII was used in some cases, but the details are not described.

### Comparative Example 2: Evaluation of Affinity of Each VL-κ-Binding Domain with Deletion of N-Terminal Region for VL-κ

With respect to each VL-κ-binding domain with a deletion of the N-terminal region prepared in Comparative example 1, the affinity for totally six IgG-Fab prepared in Example 2(1) was analyzed similarly to the condition of Example 2(2). The analysis result is shown in Table 3 and Table 4.

TABLE 3

| | kₒₙ (×10⁵ [M⁻¹s]) | k_{off} (×10⁻² [s⁻¹]) | K_{A} (×10⁶ [M⁻¹]) |
|---|---|---|---|
| LB1t-Wild.1d | | | |
| aRSV-Fab | 2.5 | 5.7 | 4.4 |
| aTNFa-Fab | 0.1 | 48.1 | 0.03 |
| aEGFR-Fab | 0.2 | 26.1 | 0.1 |
| aHER2-Fab | 50.9 | 2.5 | 201.2 |
| aIgE-Fab | 107.0 | 1.0 | 1119.2 |
| aRANKL-Fab | 0.1 | 23.3 | 0.1 |
| LB2t-Wild.1d | | | |
| aRSV-Fab | 1.2 | 12.2 | 1.0 |
| aTNFa-Fab | N.D. | N.D. | N.D. |
| aEGFR-Fab | N.D. | N.D. | N.D. |
| aHER2-Fab | 44.9 | 2.5 | 182.5 |
| aIgE-Fab | N.D. | N.D. | N.D. |
| aRANKL-Fab | 0.01 | 34.3 | 0.004 |
| LB3t-Wild.1d | | | |
| aRSV-Fab | 1.0 | 6.7 | 1.4 |
| aTNFa-Fab | 0.2 | 30.5 | 0.1 |
| aEGFR-Fab | 0.3 | 21.9 | 0.1 |
| aHER2-Fab | 3.8 | 1.4 | 28.0 |
| aIgE-Fab | 18.2 | 0.5 | 376.8 |
| aRANKL-Fab | 0.02 | 19.7 | 0.01 |
| LB4t-Wild.1d | | | |
| aRSV-Fab | 4.7 | 3.4 | 13.8 |
| aTNFa-Fab | 0.7 | 23.9 | 0.3 |
| aEGFR-Fab | 1.1 | 18.7 | 0.6 |
| aHER2-Fab | 27.8 | 1.3 | 220.6 |
| aIgE-Fab | 43.3 | 0.4 | 1077.1 |
| aRANKL-Fab | 1.5 | 26.1 | 0.6 |

TABLE 4

| | kₒₙ (×10⁵ [M⁻¹s]) | k_{off} (×10⁻² [s⁻¹]) | K_{A} (×10⁶ [M⁻¹]) |
|---|---|---|---|
| LC1t-Wild.1d | | | |
| aRSV-Fab | 2.0 | 8.6 | 2.3 |
| aTNFa-Fab | 0.2 | 15.2 | 0.2 |
| aEGFR-Fab | 0.1 | 7.7 | 0.2 |
| aHER2-Fab | 1.8 | 4.1 | 4.3 |
| aIgE-Fab | 5.5 | 2.0 | 27.1 |
| aRANKL-Fab | 0.1 | 8.9 | 0.1 |
| LC2t-Wild.1d | | | |
| aRSV-Fab | 2.2 | 2.4 | 9.2 |
| aTNFa-Fab | N.D. | N.D. | N.D. |
| aEGFR-Fab | N.D. | N.D. | N.D. |
| aHER2-Fab | 30.4 | 1.3 | 233.8 |
| aIgE-Fab | N.D. | N.D. | N.D. |
| aRANKL-Fab | N.D. | N.D. | N.D. |
| LC3t-Wild.1d | | | |
| aRSV-Fab | 1.8 | 2.1 | 8.7 |
| aTNFa-Fab | N.D. | N.D. | N.D. |
| aEGFR-Fab | N.D. | N.D. | N.D. |
| aHER2-Fab | 2.2 | 1.2 | 18.7 |
| aIgE-Fab | N.D. | N.D. | N.D. |
| aRANKL-Fab | N.D. | N.D. | N.D. |
| LC4t-Wild.1d | | | |
| aRSV-Fab | 3.4 | 1.7 | 19.3 |
| aTNFa-Fab | 0.1 | 39.7 | 0.02 |
| aEGFR-Fab | 0.2 | 25.3 | 0.1 |
| aHER2-Fab | 25.2 | 0.6 | 405.8 |
| aIgE-Fab | 52.3 | 0.1 | 4184.0 |
| aRANKL-Fab | 0.03 | 18.2 | 0.02 |

As the result shown in Tables 3 and 4, the B1 to B4 and C1 to C4 domains derived from Peptostreptococcus magnus have high affinity for the specific Fab region in some cases, but do not have an affinity or have very low affinity for the other Fab region. On the one hand, as the result shown in Table 2, the B5 domain derived from Peptostreptococcus magnus according to one or more embodiments of the present invention has strong affinity for all of the tested Fab regions on the whole. Thus, it was demonstrated that the B5 domain of one or more embodiments of the present invention is particularly usable for purifying a peptide containing a Fab region.

### Example 3: Preparation of Tetramer of B5 Domain of PpL 312 (LB5t-Wild.4d)

The amino acid sequence of SEQ ID NO: 26 (“LB5t-Wild.4d”) was designed. The sequence was composed of the 4 amino acid sequences of B5 domain of SEQ ID NO: 16 linked by the amino acid sequence between VL-κ-binding domains in PpL 312 of SEQ ID NO: 1. The base sequence of SEQ ID NO: 27 which encoded the peptide was designed by conducting reverse translation on the basis of the amino acid sequence of LB5t-Wild.4d (SEQ ID NO: 26). The DNA (SEQ ID NO: 28) which had the base sequence (SEQ ID NO: 27), PstI recognition site at 5′ end and XbaI recognition site at 3′ end was synthesized as an artificial synthetic gene by outsourcing to Eurofins Genomics K. K. The expression plasmid after subcloning was digested by restriction enzyme PstI and XbaI manufactured by Takara Bio Inc. to obtain a DNA fragment. An expression vector pNCMO2 for Brevibacillus Expression System manufactured by Takara Bio Inc. was digested by the same restriction enzyme, and the obtained DNA fragment was ligated thereto to obtain an expression vector into which DNA encoding the amino acid sequence of LB5t-Wild.4d was inserted. The ligation reaction was performed by using a ligation reagent (“Ligation high” manufactured by TOYOBO CO., LTD.) in accordance with the protocol attached to the product, and Escherichia coli JM109 strain manufactured by Takara Bio Inc. was used for preparing the plasmid. The base sequence of each expression plasmid DNA was confirmed by using DNA sequencer 3130xl Genetic Analyzer manufactured by Applied Biosystems. A sequencing PCR reaction of each plasmid DNA was performed by using BigDye Terminator v.1.1 Cycle Sequencing Kit manufactured by Applied Biosystems in accordance with the protocol attached to the product, and the obtained sequencing product was purified by using a plasmid purification kit (“BigDye XTerminator Purification Kit” manufactured by Applied Biosystems) in accordance with the protocol attached to the product to be used for sequence analysis.

Brevibacillus choshinensis SP3 strain manufactured by Takara Bio Inc. was transformed by the obtained plasmid, and the genetically modified bacterium which produced and secreted LB5t-Wild.4d was cultivated. The genetically modified bacterium was cultivated in 30 mL of A culture medium (polypeptone 3.0%, yeast extract 0.5%, glucose 3%, magnesium sulfate 0.01%, ferric sulfate 0.001%, manganese chloride 0.001%, zinc chloride 0.0001%) containing 60 μg/mL of neomycin with shaking at 30° C. for 3 days. After the cultivation, the culture medium was subjected to centrifugation at 15,000 rpm and at 25° C. for 5 minutes to remove the bacterial body.

LB5t-Wild.4d was purified from the obtained culture supernatant by cation exchange chromatography for which a cation exchange carrier (“UnoSphere S” manufactured by Bio-Rad) was used. A column (“Tricorn 10/200” manufactured by GE Healthcare Bioscience) was filled with the UnoSphere S. Specifically, sodium acetate was added to the culture supernatant at a final concentration of 50 mM, and the pH thereof was adjusted to 4.0 by acetic acid. The UnoSphere S column was equilibrated by buffer A for cation exchange (50 mM CH₃COOH—CH₃COONa, pH 4.0), and the culture supernatant was added into the column. After the column was washed by the buffer A for cation exchange, LB5t-Wild.4d was eluted to be obtained by using salt concentration gradient with the buffer A for cation exchange and buffer B for cation exchange (50 mM CH₃COOH—CH₂COONa, 1 M NaCl, pH 4.0). Then, the LB5t-Wild.4d was purified by anion exchange chromatography for which an anion exchange carrier (“Nuvia Q” manufactured by Bio-Rad) was used. A column (“Tricorn 10/200” manufactured by GE Healthcare Bioscience) was filled with the Nuvia Q. Specifically, the obtained LB5t-Wild.4d solution was dialyzed by using a buffer A for anion exchange (50 mM Tris-HCl, pH 8.0). The Nuvia Q column was equilibrated by buffer A for anion exchange, and the solution was added into the column. After the column was washed by the buffer A for anion exchange, LB5t-Wild.4d was eluted to be obtained by using salt concentration gradient with the buffer A for anion exchange and buffer B for anion exchange (50 mM Tris-HCl, 1.0 M NaCl, pH 8.0). The obtained LB5t-Wild.4d was dialyzed by using ultrapure water, and an aqueous solution containing the LB5t-Wild.4d only was obtained as a final purified sample. The above-described protein purification by chromatography with a column was performed by using a chromatography system (“AKTAavant 25 system” manufactured by GE Healthcare Bioscience).

### Example 4: Preparation of Carrier on which Tetramer of B5 Domain of PpL 312 was Immobilized

The obtained LB5t-Wild.4d prepared in Example 3 was immobilized on a commercially available agarose carrier. For such immobilization, the combination of the reactive amino acid residue of LB5t-Wild.4d and maleimide was used.

Specifically, first, 1.5 mL-gel of commercially available NHS-activated carrier (“NHS Activated Sepharose 4 Fast Flow” manufactured by GE Healthcare Bioscience) was transferred on a glass filter, and isopropanol as a preservation liquid was removed by suction, and then the carrier was washed by ice cooled 1 mM hydrochloric acid (5 mL). Next, after the carrier was washed by 5 mL of a coupling buffer (20 mM NaH₂PO₄—Na₂HPO₄, 150 mM sodium chloride, pH 7.2), the washed carrier was collected with suspending in the coupling buffer and transferred into a centrifuge tube. N-[ε-Maleimidocaproic acid]hydrazide.TFA (EMCH, manufactured by Thermo Fisher Scientific Inc.) was dissolved in the coupling buffer to obtain 10 mM solution, and the solution was added into the centrifuge tube with the carrier for the reaction at 25° C. for 1 hour. Then, the carrier was transferred on a glass filter, and washed with 10 mL of washing buffer A (0.5 M ethanolamine, 0.5 M sodium chloride, pH 7.2), 10 mL of the coupling buffer and 10 mL of the washing buffer A in this order, and left to stand at 25° C. for 15 minutes. The carrier was further washed with the coupling buffer (10 mL). According to the preceding procedure, maleimide was bound to the carrier.

Next, LB5t-Wild.4d was immobilized on the carrier bound by maleimide. Before the immobilization, LB5t-Wild.4d was reduced under the condition of 100 mM DTT. Further, DTT was removed by using a desalting column (“HiTrap Desalting” GE Healthcare Bioscience) and the buffer was exchanged to a coupling buffer. The carrier bound by maleimide was transferred into a centrifuge tube, and the LB5t-Wild.4d solution was added thereto for the reaction at 25° C. for 2 hours. Then, the reacted carrier was transferred on a glass filter, and washed with 7 mL of the coupling buffer to recover the unreacted LB5t-Wild.4d. Thereafter, the carrier was washed with 10 mL of a washing buffer B (50 mM L-cysteine, 100 mM NaH₂PO₄—Na₂HPO₄, 0.5 M sodium chloride, pH 7.2), 10 mL of the coupling buffer and 10 mL of the washing buffer B in this order, and then the carrier was left to stand at 25° C. for 15 minutes. After the carrier was further washed with 10 mL of the coupling buffer, 10 mL of ultrapure water and 10 mL of 20% ethanol, the carrier was suspended in 20% ethanol and collected to obtain LB5t-Wild.4d-immobilized carrier.

An absorbance of the recovered unreacted LB5t-Wild.4d at 280 nm was measured by using a spectrometer, and an amount of the unreacted LB5t-Wild.4d was calculated on the basis of the absorption coefficient calculated from the amino acid sequence. The amount of the immobilized LB5t-Wild.4d was calculated on the basis of the difference between the amount of the originally-used LB5t-Wild.4d and the determined amount of the unreacted LB5t-Wild.4d, and the ligand density was further calculated on the basis of the volume of the carrier. The ligand density of the prepared carrier as an experimental product 1 and the ligand density of a commercially available Protein L carrier (“HiTrap Protein L” GE Healthcare Bioscience) used as Comparative example 3 are shown in Table 5.

TABLE 5

| Carrier | Ligand density (mg/mL-gel) |
|---|---|
| Example 4 | 10 |
| Comparative example 3 | 10 |

### Example 5: Confirmation of Adsorption of Polyclonal Fab on LB5t-Wild.4d-Immobilized Carrier

In order to evaluate the binding ability of the LB5t-Wild.4d-immobilized carrier prepared in Example 4, it was confirmed whether human polyclonal Fab was adsorbed. As human polyclonal Fab, polyclonal Fab derived from human polyclonal antibody (“γ-globulin” Nihon Pharmaceutical Co., Ltd.) was prepared. The polyclonal Fab was prepared in accordance with Example 2(1). Since the human polyclonal antibody contained a component which was not adsorbed on a Protein A carrier, IgG as an adsorption component was collected by affinity chromatography using KANEKA KanCapA™ column (KANEKA Corporation) before the digestion by papain and the collected IgG was digested by papain.

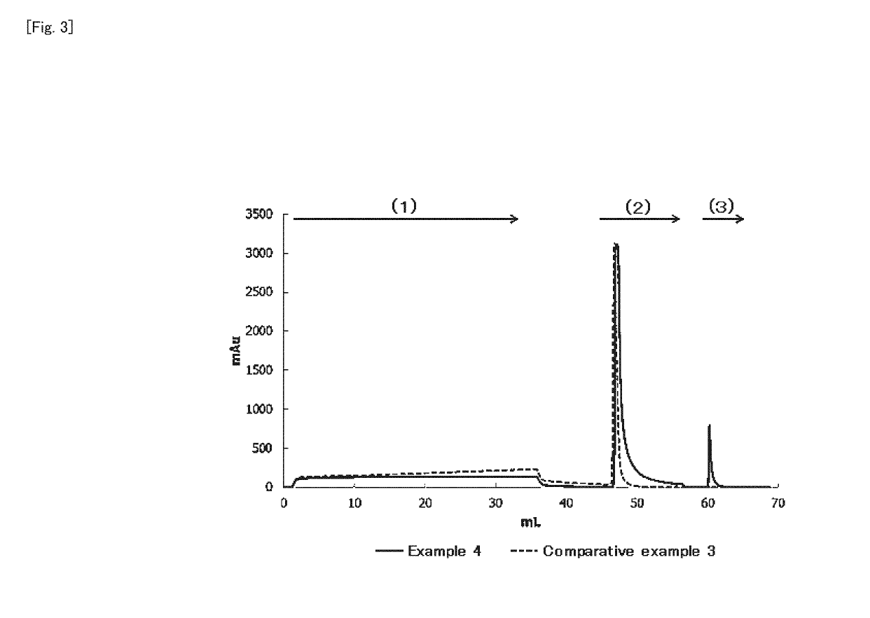

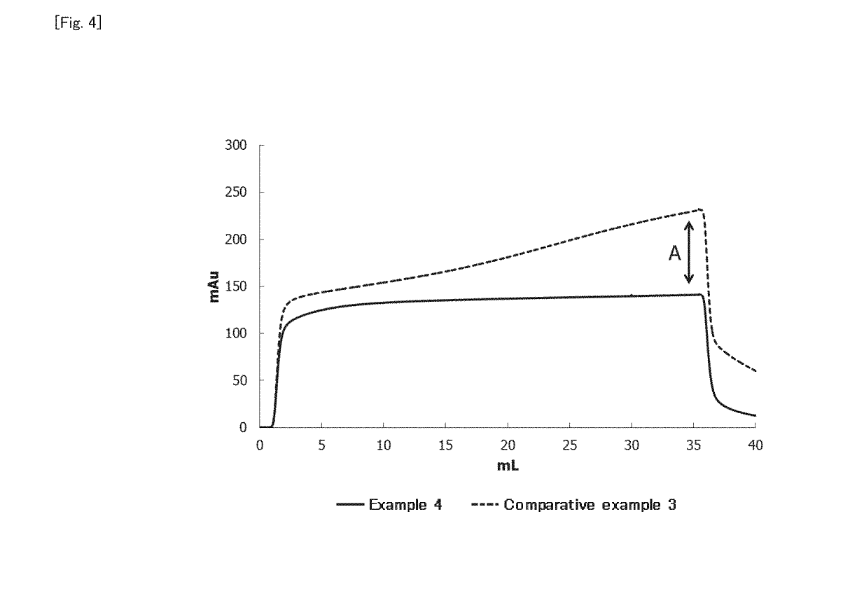

Hereinafter, the specific procedure of the method for confirming the binding ability of LB5t-Wild.4d-immobilized carrier is described. Tricorn 5/50 column (GE Healthcare Bioscience) was filled with 1 mL-gel of the carrier, and connected to chromatography system AKTAavant 25. The column was equilibrated by flowing 3 CV of an equilibrating buffer (20 mM NaH₂PO₄—Na₂HPO₄, 150 mM sodium chloride, pH 7.4) through the column at a flow rate of 0.25 mL/min. Then, 35 mL of 1 mg/mL human polyclonal Fab solution was flown at a flow rate of 0.25 mL/min. Next, 10 CV of the equilibrating buffer was flown at a flow rate of 0.25 mL/min, and subsequently 10 CV of an elution buffer (50 mM citric acid, pH 3.0) was flown to elute human polyclonal Fab. Further, after 3 CV of the equilibrating buffer was flown at a flow rate of 0.25 mL/min, 5 CV of a strong washing buffer (50 mM citric acid, pH 2.5) was flown and finally 5 CV of the equilibrating buffer was flown. As Comparative example 3, a similar procedure was performed with respect to commercial available Protein L carrier (“HiTrap Protein L” GE Healthcare Bioscience). The obtained chromatography charts are demonstrated as FIG. 3, and the enlarged chromatography charts of the step for applying human polyclonal Fab are demonstrated as FIG. 4.

The peak area of the elution fraction (2) of FIG. 3 in the case where the carrier of Example 4 was used was clearly larger than that in the case where the carrier of Comparative example 3 was used. In addition, as the difference A in FIG. 4, which is enlarged part corresponding to the step (1) for applying human polyclonal Fab, a leakage tends to continuously increase from the carrier of Comparative example 3 at the time of the addition of human polyclonal Fab; on the one hand, a leakage amount from the carrier of Example 3 is constant. There is a Fab which contains a λ chain variable region among human polyclonal Fab, and such a Fab containing a λ chain variable region is not adsorbed on the carriers of Comparative example 3 and Example 4 and leaked at the time of the addition of human polyclonal Fab. Furthermore, it is indicated by the difference A that there is a Fab which contains a κ chain variable region and which is adsorbed on the carrier of Example 4 but is not adsorbed on the carrier of Comparative example 3. Thus, though the amounts of the human polyclonal Fab which added to each of the carrier were the same, the elution peak in the case of Example 4 was larger than that in the case of Comparative example 3. In other words, the amount of Fab which was adsorbed on the carrier of Example 4 was larger.

It was demonstrated from the result that the kind of an antibody fragment which can be purified by the affinity separation matrix on which the κ chain variable region-binding peptide of one or more embodiments of the present invention is immobilized can be expanded.

### Example 6: Evaluation of Binding Ability of LB5t-Wild.4d-Immobilized Carrier to Monoclonal Fab

With respect to the LB5t-Wild.4d-immobilized carrier prepared in Example 4, a binding ability to monoclonal Fab was evaluated. With respect to monoclonal Fab, aTNFa-Fab prepared in Example 2(1) was dissolved in an equilibrating buffer (20 mM NaH₂PO₄—Na₂HPO₄, 150 mM sodium chloride, pH 7.4) at a concentration of 1 mg/mL solution, and the solution was used.

A column (“Tricorn 5/50 column” GE Healthcare Bioscience) was filled with 1 mL-gel of the carrier, and connected to chromatography system AKTAavant 25. The column was equilibrated by flowing 3 CV of an equilibrating buffer (20 mM NaH₂PO₄—Na₂HPO₄, 150 mM sodium chloride, pH 7.4) at a flow rate of 0.25 mL/min. Then, the aTNFa-Fab solution was flown at a flow rate of 0.25 mL/min until a monitoring absorbance exceeded 55% of 100% Abs₂₈₀. Next, 10 CV of the equilibrating buffer was flown at a flow rate of 0.25 mL/min, and subsequently 3 CV of an elution buffer (50 mM citric acid, pH 2.5) was flown to elute aTNFa-Fab. The total amount of the aTNFa-Fab solution which was flown until a monitoring absorbance exceeded 55% of 100% Abs₂₈₀ was defined as 55% DBC, i.e. quasi-static binding capacity, to aTNFa-Fab. A similar procedure was performed with respect to commercial available Protein L carrier (“HiTrap Protein L” GE Healthcare Bioscience) as Comparative example 3. The measurement result is shown in Table 6.

TABLE 6

| Carrier | 55% DBC (mg/mL-gel) |
|---|---|
| Example 4 | 48.6 |
| Comparative example 3 | 4.8 |

As the result shown in Table 6, 55% DBC to aTNFa-Fab of the carrier of Example 4 is much larger than that of Comparative example 3. It was demonstrated from the result that Fab containing a κ chain variable region is hardly adsorbed on the carrier of Comparative example 3 but can be adsorbed to be purified on the affinity separation matrix on which the κ chain variable region-binding peptide of one or more embodiments of the present invention is immobilized as a ligand.

### Example 7: Evaluation of Resistance of LB5t-Wild.1d to Alkali

The dialyzed LB5t-Wild.1d was dissolved in water to obtain 0.04 mL of 40 μM aqueous solution. To the aqueous solution, 0.02 mL of 150 mM sodium hydroxide aqueous solution was added so that the final concentration of sodium hydroxide became 50 mM. After the mixture was incubated at 25° C. for 2 hours, the mixture was neutralized by using 0.02 mL of 50 mM citric acid (pH 2.4). For a comparison, the above-described alkali and acid were preliminarily mixed in the same ratio to obtain a solution, the solution was added to the above-described sample before the alkaline treatment, and the mixture was similarly incubated at 25° C. for 2 hours. It was confirmed by pH test paper whether the mixture was neutralized. For a repeatability, one experiment of the above-described series of alkaline treatment procedure was further conducted.

The measurement was performed by immobilizing about 5000 RU of aRSV-Fab on a sensor chip CM5 and adjusting a flow rate to 10 μL/min in Biacore 3000. By adjusting an immobilized amount to high as 5000 RU or more and a flow rate to be slow, a detection sensitivity and a dependence on an analyte concentration are improved. In other words, under the condition where mass transport is limited, the dependence of a binding response on an affinity is decreased and the dependence on a concentration is relatively increased.

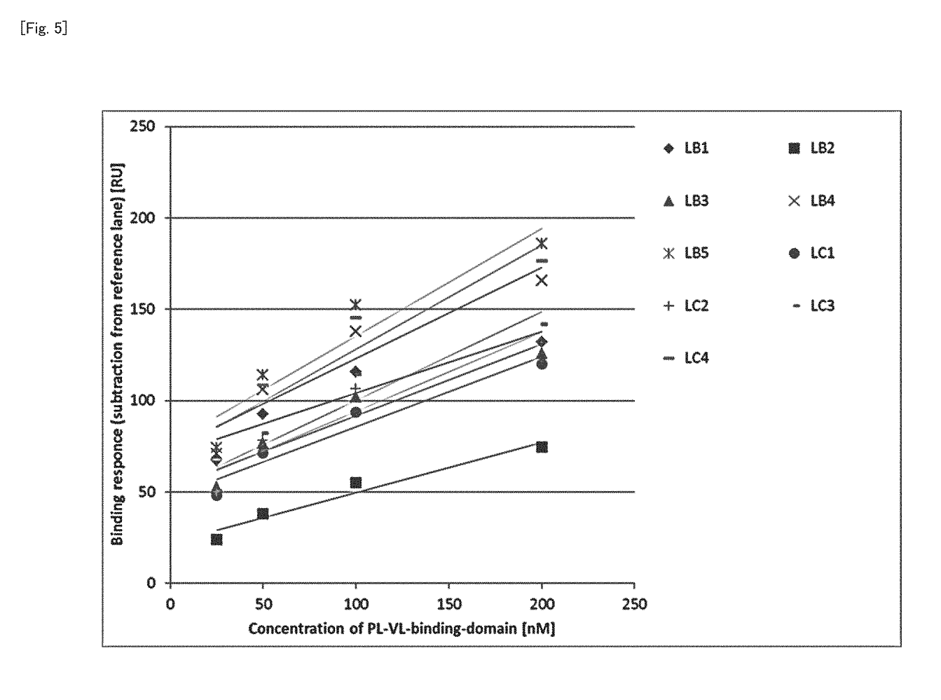

LB5t-Wild.1d before the alkaline treatment was dissolved in the running buffer to obtain protein solutions having the concentrations of 50 nM, 100 nM or 200 nM. The solution was added on the sensor chip at a flow rate of 10 μL/min for 2 minutes. Binding response curves at the time of addition (association phase, for 2 minutes) and after the addition (dissociation phase, for 2 minutes) were sequentially obtained at a measurement temperature of 25° C. After each measurement, about 20 mM NaOH was added for washing. A binding response 1 minute after the addition (resonance unit value of a binding response curve) was plotted on a vertical axis of a graph, and the added analyte concentration at the addition was plotted on an abscissa axis. The graph is shown as FIG. 5. In the concentration range of the evaluation system, a bonding response is somewhat proportional to an analyte concentration. As the graphs, the increase degrees of the binding responses to the analyte concentrations are different depending on the kind of the domain. In this evaluation system, a mere ratio of responses before and after the alkaline treatment was not used, but after correcting to convert to concentration, a residual binding activity was calculated.

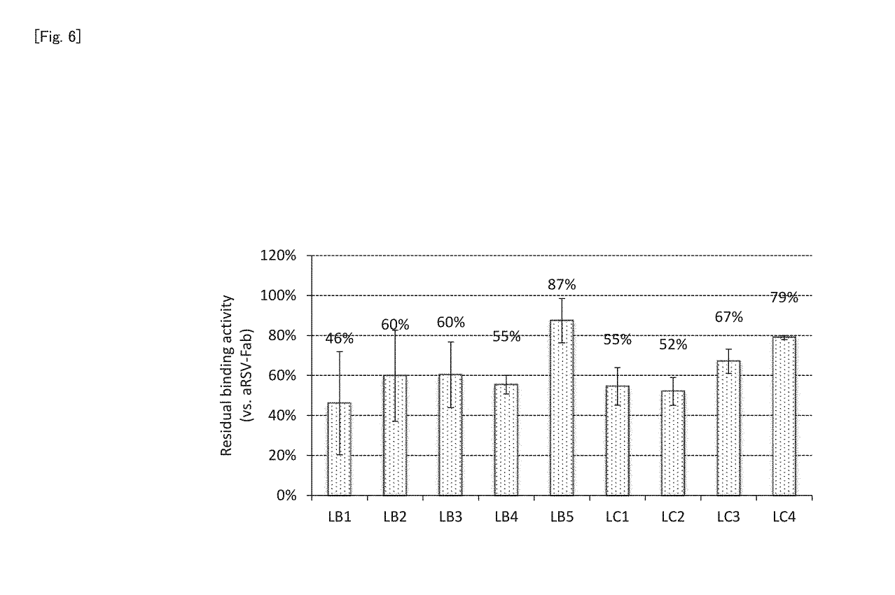

A concentration of LB5t-Wild.1d after the alkaline treatment was also adjusted to 200 nM by using the running buffer, and the solution was added to the sensor tip at a flow rate of 10 μL/min for 2 minutes similarly to the above to measure the binding response 1 minute after the addition. An analyte concentration to the binding response value after the alkaline treatment was calculated on the basis of the previously obtained binding response value at 200 nM before the alkaline treatment and the approximate curves shown in FIG. 4. In addition, the concentration was regarded as a concentration of variant of which binding activity was maintained, and a concentration ratio to the concentration before the alkaline treatment as 100% was calculated as a residual binding activity. With respect to LB5t-Wild.1d, an average value and a deviation were obtained from the residual binding activity values data of totally N=4, and are graphically shown as FIG. 6. It was found from the residual binding activity values that the resistance of B5 domain to an alkali is superior to other domains. Such a property cannot be found on the basis of a general biological perspective, and is considered to be particularly useful for regenerating the affinity separation matrix to be repeatedly used.

### Comparative Example 3: Evaluation of Resistance of Various VL-κ-Binding Domains with Deletion of N-Terminal Region to Alkali

The resistance of various VL-κ-binding domains with a deletion of a N-terminal region prepared in Comparative example 1 to an alkali was evaluated in a similar condition to Example 3. The analysis result is shown in FIG. 5 and FIG. 6.

### Example 8: Evaluation of Resistance of LB5t-Wild.4d-Immibilized Carrier to Alkali

The resistance of the LB5t-Wild.4d-immibilized carrier prepared in Example 4 to an alkali was evaluated by comparing the binding abilities to monoclonal Fab before and after the alkaline washing. The aIgE-Fab prepared in Example 2(1) was dissolved in the equilibrating buffer to obtain 1 mg/mL solution. The solution was used, and a flow rate was adjusted to 0.33 mL/min. By a similar method to Example 6, 55% DBC of each carrier to aIgE-Fab before contacting with 50 mM NaOH was measured. Then, 9.9 mL of 50 mM NaOH was flown to be contacted with the carrier for 30 minutes and the equilibrating buffer was flown. After the cycle was repeated 5 times, 55% DBC was measured again.

As a result, 55% DBC before the alkaline washing was 23.6 mg/mL and 55% DBC after the alkaline washing was 21.9 mg/mL, and 92.8% of a binding capability to Fab was maintained even after an alkaline treatment. Thus, since 90% or more of 55% DBC was maintained even after a washing procedure with 50 mM NaOH for 30 minutes was performed 5 times, it was demonstrated that the affinity separation matrix of one or more embodiments of the present invention can withstand repeated use with alkaline washing.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method for producing a protein, comprising:
adsorbing a protein comprising a κ chain variable region on an insoluble carrier of an affinity separation matrix by contacting a liquid sample comprising the protein with the affinity separation matrix;
washing the affinity separation matrix to remove impurities;
separating the protein from the affinity separation matrix by using an acidic buffer; and
regenerating the affinity separation matrix by using an alkaline aqueous solution after the protein is separated from the affinity separation matrix,
wherein the insoluble carrier comprises a ligand immobilized on the insoluble carrier,
wherein the ligand is a κ chain variable region-binding peptide comprising B5 domain of Protein L derived from Peptostreptococcus magnus 312 strain or a variant of the B5 domain, and
wherein the adsorbing, the washing, and the separating are repeated 3 or more times.

2. The method according to claim 1, wherein the regenerating is also repeated 3 or more times after the separating.

3. The method according to claim 1,
wherein an amino acid sequence of the B5 domain is an amino acid sequence of SEQ ID NO: 7 or SEQ ID NO: 16, and
wherein an amino acid sequence of the variant of the B5 domain is selected from the group consisting of:
an amino acid sequence of SEQ ID NO: 7 or SEQ ID NO: 16 comprising a total of 1 to 10 amino acid deletions, substitutions and/or additions, wherein the amino acid sequence has a binding ability to the κ chain variable region; and
an amino acid sequence having a sequence identity of 85% or more with the amino acid sequence of SEQ ID NO: 7 or SEQ ID NO: 16, wherein the amino acid sequence has a binding ability to the κ chain variable region.

4. The method according to claim 3,
wherein the κ chain variable region-binding peptide comprises the variant of the B5 domain, and
wherein the amino acid sequence of the variant of the B5 domain is an amino acid sequence corresponding to SEQ ID NO: 7, wherein the 17^{th} position is Glu, the 19^{th} position is Ile, the 20^{th} position is Tyr, the 22^{nd} position is Glu, the 25^{th} position is Thr, the 26^{th} position is Val, the 30^{th} position is Thr, the 50^{th} position is Ser and the 53^{rd} position is His.

5. The method according to claim 3,
wherein the κ chain variable region-binding peptide comprises the variant of the B5 domain, and
wherein the amino acid sequence of the variant of the B5 domain is an amino acid sequence corresponding to SEQ ID NO: 16, wherein the 7^{th} position is Glu, the 9^{th} position is Ile, the 10^{th} position is Tyr, the 12^{th} position is Glu, the 15^{th} position is Thr, the 16^{th} position is Val, the 20^{th} position is Thr, the 40^{th} position is Ser and the 43^{rd} position is His.

6. The method according to claim 3, wherein the ligand is a multimer of the B5 domain or the variant.


SEQUENCE LISTING (28 sequences)

SEQ ID NO: 1 - protein, 719 aa, Peptostreptococcus magnus
MAALAGAIVVTGGVGSYAADEPIDLEKLEEKRDKENVGNLPKFDNEVKDGSENPMAKYPDFDDEASTRFETENNEFEEKKVVSDNFFDQSEHPFVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGKEEPEETPEKPEVQDGYASYEEAEAAAKEALKNDDVNKSYTIRQGADGRYYYVLSPVEAEEEKPEAQNGYATYEEAEAAAKKALENDPINKSYSIRQGADGRYYYVLSPVEAETPEKPVEPSEPSTPDVPSNPSNPSTPDVPSTPDVPSNPSTPEVPSNPSTPGNEEKPGNEQKPGNEQKPGNEQKPGNEQKPGNEQKPDQPSKPEKEENGKGGVDSPKKKEKAALPKAGSEAEILTLAAASLSSVAGAFISLKKRK

SEQ ID NO: 2 - protein, 992 aa, Peptostreptococcus magnus
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 3 - protein, 76 aa, Peptostreptococcus magnus
KEETPETPETDSEEEVTIKANLIFANGSTQTAEFKGTFEKATSEAYAYADTLKKDNGEYTVDVADKGYTLNIKFAG

SEQ ID NO: 4 - protein, 72 aa, Peptostreptococcus magnus
KEKTPEEPKEEVTIKANLIYADGKTQTAEFKGTFEEATAEAYRYADALKKDNGEYTVDVADKGYTLNIKFAG

SEQ ID NO: 5 - protein, 72 aa, Peptostreptococcus magnus
KEKTPEEPKEEVTIKANLIYADGKTQTAEFKGTFEEATAEAYRYADLLAKENGKYTVDVADKGYTLNIKFAG

SEQ ID NO: 6 - protein, 72 aa, Peptostreptococcus magnus
KEKTPEEPKEEVTIKANLIYADGKTQTAEFKGTFAEATAEAYRYADLLAKENGKYTADLEDGGYTINIRFAG

SEQ ID NO: 7 - protein, 73 aa, Peptostreptococcus magnus
KKVDEKPEEKEQVTIKENIYFEDGTVQTATFKGTFAEATAEAYRYADLLSKEHGKYTADLEDGGYTINIRFAG

SEQ ID NO: 8 - protein, 71 aa, Peptostreptococcus magnus
KETPEPEEEVTIKANLIFADGSTQNAEFKGTFAKAVSDAYAYADALKKDNGEYTVDVADKGLTLNIKFAGK

SEQ ID NO: 9 - protein, 71 aa, Peptostreptococcus magnus
KEKPEEPKEEVTIKVNLIFADGKTQTAEFKGTFEEATAKAYAYADLLAKENGEYTADLEDGGNTINIKFAG

SEQ ID NO: 10 - protein, 74 aa, Peptostreptococcus magnus
KETPETPEEPKEEVTIKVNLIFADGKIQTAEFKGTFEEATAKAYAYANLLAKENGEYTADLEDGGNTINIKFAG

SEQ ID NO: 11 - protein, 75 aa, Peptostreptococcus magnus
KETPETPEEPKEEVTIKVNLIFADGKTQTAEFKGTFEEATAEAYRYADLLAKVNGEYTADLEDGGYTINIKFAGK

SEQ ID NO: 12 - protein, 63 aa, Peptostreptococcus magnus
EEVTIKANLIFANGSTQTAEFKGTFEKATSEAYAYADTLKKDNGEYTVDVADKGYTLNIKFAG

SEQ ID NO: 13 - protein, 63 aa, Peptostreptococcus magnus
EEVTIKANLIYADGKTQTAEFKGTFEEATAEAYRYADALKKDNGEYTVDVADKGYTLNIKFAG

SEQ ID NO: 14 - protein, 63 aa, Peptostreptococcus magnus
EEVTIKANLIYADGKTQTAEFKGTFEEATAEAYRYADLLAKENGKYTVDVADKGYTLNIKFAG

SEQ ID NO: 15 - protein, 63 aa, Peptostreptococcus magnus
EEVTIKANLIYADGKTQTAEFKGTFAEATAEAYRYADLLAKENGKYTADLEDGGYTINIRFAG

SEQ ID NO: 16 - protein, 63 aa, Peptostreptococcus magnus
EQVTIKENIYFEDGTVQTATFKGTFAEATAEAYRYADLLSKEHGKYTADLEDGGYTINIRFAG

SEQ ID NO: 17 - protein, 64 aa, Peptostreptococcus magnus
EEVTIKANLIFADGSTQNAEFKGTFAKAVSDAYAYADALKKDNGEYTVDVADKGLTLNIKFAGK

SEQ ID NO: 18 - protein, 63 aa, Peptostreptococcus magnus
EEVTIKVNLIFADGKTQTAEFKGTFEEATAKAYAYADLLAKENGEYTADLEDGGNTINIKFAG

SEQ ID NO: 19 - protein, 63 aa, Peptostreptococcus magnus
EEVTIKVNLIFADGKIQTAEFKGTFEEATAKAYAYANLLAKENGEYTADLEDGGNTINIKFAG

SEQ ID NO: 20 - protein, 64 aa, Peptostreptococcus magnus
EEVTIKVNLIFADGKTQTAEFKGTFEEATAEAYRYADLLAKVNGEYTADLEDGGYTINIKFAGK

SEQ ID NO: 21 - DNA, 195 bp, Artificial Sequence
  synthetic DNA
tccgaacaggttaccattaaagagaacatctactttgaagatggcacggtacagactgccacgttcaaaggtacgtttgcggaagctactgcagaagcttaccgctatgcggacctgctctcgaaagagcatggcaaatacacagcggatcttgaagatggaggttacacaatcaatattcgcttcgccggctaa

SEQ ID NO: 22 - DNA, 66 bp, Artificial Sequence
  synthetic DNA
cgtggatccgaacaggttaccattaaagagaacatctactttgaagatggcacggtacagactgcc

SEQ ID NO: 23 - DNA, 54 bp, Artificial Sequence
  synthetic DNA
gcggtaagcttctgcagtagcttccgcaaacgtacctttgaacgtggcagtctg

SEQ ID NO: 24 - DNA, 63 bp, Artificial Sequence
  synthetic DNA
gcagaagcttaccgctatgcggacctgctctcgaaagagcatggcaaatacacagcggatctt

SEQ ID NO: 25 - DNA, 60 bp, Artificial Sequence
  synthetic DNA
gatgaattcttagccggcgaagcgaatattgattgtgtaacctccatcttcaagatccgc

SEQ ID NO: 26 - protein, 302 aa, Peptostreptococcus magnus
KEETPETPETDSEEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKKVDEKPEEC

SEQ ID NO: 27 - DNA, 906 bp, Artificial Sequence
  synthetic DNA
aaagaagaaaccccggaaaccccagaaaccgactctgaagaacaggtcacaatcaaggaaaacatttattttgaagatggtaccgtgcaaaccgcaacttttaaaggcacattcgctgaagctacggcggaggcgtatcgttacgcggacttactgtcgaaagaacatggtaaatataccgccgatctggaagatggcggctataccattaacattcgcttcgcaggcaaagagaagacccctgaagaaccgaaggaacaggtgaccattaaagagaatatctatttcgaagacggtactgtacagaccgcgacgtttaaaggaacctttgccgaagccacggccgaggcttatcgctacgccgatcttctgtccaaagagcatggcaagtacacggcggacctggaggatgggggttacactatcaacattcggtttgcgggtaaggaaaaaacaccggaagaacccaaagaacaagtcactatcaaagagaacatttactttgaggacggcacggttcaaacggcaaccttcaaagggacgtttgcggaagcgacagcagaagcctaccgctatgctgatctactgagcaaagaacacggcaagtatactgcggatttggaagacggaggttacaccatcaatatccgattcgcgggcaaagaaaaaactccagaagagccgaaagagcaggttaccataaaagaaaatatttacttcgaagatggcaccgtacagacggcaacattcaagggtacctttgctgaggcaaccgccgaagcataccgttatgccgacctcctgagtaaagagcacggaaaatatacggcggatttagaggatggcggttatacgatcaatattcgttttgccgggaaaaaagtggatgaaaaaccggaggaatgc

SEQ ID NO: 28 - DNA, 923 bp, Artificial Sequence
  synthetic DNA
ctgcaggaaaagaagaaaccccggaaaccccagaaaccgactctgaagaacaggtcacaatcaaggaaaacatttattttgaagatggtaccgtgcaaaccgcaacttttaaaggcacattcgctgaagctacggcggaggcgtatcgttacgcggacttactgtcgaaagaacatggtaaatataccgccgatctggaagatggcggctataccattaacattcgcttcgcaggcaaagagaagacccctgaagaaccgaaggaacaggtgaccattaaagagaatatctatttcgaagacggtactgtacagaccgcgacgtttaaaggaacctttgccgaagccacggccgaggcttatcgctacgccgatcttctgtccaaagagcatggcaagtacacggcggacctggaggatgggggttacactatcaacattcggtttgcgggtaaggaaaaaacaccggaagaacccaaagaacaagtcactatcaaagagaacatttactttgaggacggcacggttcaaacggcaaccttcaaagggacgtttgcggaagcgacagcagaagcctaccgctatgctgatctactgagcaaagaacacggcaagtatactgcggatttggaagacggaggttacaccatcaatatccgattcgcgggcaaagaaaaaactccagaagagccgaaagagcaggttaccataaaagaaaatatttacttcgaagatggcaccgtacagacggcaacattcaagggtacctttgctgaggcaaccgccgaagcataccgttatgccgacctcctgagtaaagagcacggaaaatatacggcggatttagaggatggcggttatacgatcaatattcgttttgccgggaaaaaagtggatgaaaaaccggaggaatgctaatctaga